(19) United States
(12) Patent Application Publication
(10) Pub. No.: US 2025/0090386 A1
(43) Pub. Date: Mar. 20, 2025

(54) ENDOLUMINAL TREATMENT SYSTEMS, DEVICES, AND RELATED METHODS

(71) Applicant: Boston Scientific Scimed, Inc., Maple Grove, MN (US)

(72) Inventors: Rahul PRABHU, Cambridge, MA (US); Dayna M. PLEAU, South Attleboro, MA (US); Jonathan ROOT, Townsend, MA (US)

(73) Assignee: Boston Scientific Scimed, Inc., Maple Grove, MN (US)

(21) Appl. No.: 18/889,929

(22) Filed: Sep. 19, 2024

Related U.S. Application Data
(60) Provisional application No. 63/583,974, filed on Sep. 20, 2023.

(51) Int. Cl. A61F 13/26; A61B 1/018; A61F 13/05; A61F 13/20

(52) U.S. Cl. CPC A61F 13/266; A61F 13/05; A61B 1/018; A61F 2013/2014

(57) ABSTRACT

A medical device may include a conduit and a porous body coupled to a distal end of the conduit. The medical device may have a first loop extending proximally from a proximal end of the porous body. The medical device may have a second loop extending distally from a distal end of the porous body.

### CROSS-REFERENCE TO RELATED APPLICATIONS

This application claims the benefit of priority under 35 U.S.C. § 119 from U.S. Provisional Application No. 63/583,974, filed Sep. 20, 2023, which is incorporated by reference herein in its entirety.

### TECHNICAL FIELD

Various aspects of this disclosure relate generally to minimally invasive medical systems, devices, and methods. In particular, aspects of the disclosure relate to medical systems, devices, and methods for endoscopic medical procedures, such as closing a wound or otherwise treating tissue.

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical leaks, or other wounds of the GI tract. Patients with perforations, post-surgical leaks, and/or other wounds in the GI tract have high mortality rates with limited treatment options. Options include endoscopic placement of clips or stents, endoscopic sutures or sealants, or surgical re-operation. Surgery is relatively invasive and has high morbidity and mortality rates. While endoscopic stent placement is a less invasive option, placing and/or removing the stent poses unique challenges. Furthermore, the stent can migrate from the intended location and/or wall off infection at a treatment site, inhibiting drainage.

The medical systems, devices, and methods of the current disclosure may rectify some of the deficiencies described above or address other aspects of the art.

### SUMMARY

Examples of this disclosure relate to, among other things, systems, devices, and methods for performing one or more medical procedures with medical devices. Specifically, this disclosure includes medical systems, devices, and methods for delivering and/or removing a device, such as, for example, an endoluminal vacuum therapy device. Each of the examples disclosed herein may include one or more of the features described in connection with any of the other disclosed examples.

According to one aspect, a medical device may comprise a conduit and a porous body coupled to a distal end of the conduit. A first loop may extend proximally from a proximal end of the porous body. A second loop may extend distally from a distal end of the porous body.

The medical device may include one or more of the following features. In a first configuration, the porous body may have a first length and a first width. In a second configuration, the porous body may have a second length and a second width. The second length may be greater than the first length. The second width may be less than the first width. In some examples, to transition the porous body from the first configuration to the second configuration, the first loop may be pulled proximally.

In some aspects, the first loop may define a proximal end of an elongate body. The second loop may define a distal end of the elongate body. A center portion may extend between each of the first loop and the second loop. Each of the first loop and the second loop may be integrally formed with the center portion in some examples. The center portion may extend through at least a portion of the porous body.

In some aspects, the center portion may include a plurality of turns. The plurality of turns may extend circumferentially around at least a portion of the porous body.

In some aspects, a netting may extend between the first loop and the second loop. The netting may extend circumferentially around at least a portion of the porous body.

In some aspects, a longitudinal axis may extend through the conduit and the porous body. The second loop may be offset from the longitudinal axis in some aspects.

In some aspects, at least one of the first loop or the second loop may be comprised of at least one of a thread, a wire, or a cable. At least one of the first loop or the second loop may be a suture.

In some aspects, the conduit may include a coupler. The coupler may be configured to removably couple the conduit and the porous body. The conduit may be configured to be coupled to a vacuum source. The vacuum source may be configured to provide a negative pressure to the porous body.

According to another aspect, a medical system may include a delivery device, a first medical device, and a second medical device. The delivery device may include a working channel extending therethrough. The first medical device may be movably disposed within the working channel of the delivery device. The second medical device may include a conduit, a porous body coupled to a distal end of the conduit, and an elongate body. A proximal end of the elongate body may include a first loop. A distal end of the elongate body may include a second loop.

The medical device may include one or more of the following features. In some aspects, a center portion of the elongate body may include a plurality of turns. The plurality of turns may extend circumferentially around at least a portion of the porous body. In a first configuration, the porous body may have a first length and a first width. In a second configuration, the porous body may have a second length and a second width. The second length may be greater than the first length. The second width may be less than the first width. To transition the porous body from the first configuration to the second configuration, the first loop may be pulled proximally. In some aspects, the second medical device may be movably disposed adjacent to the delivery device.

Aspects of this disclosure also include methods for performing a medical procedure. A method may include inserting a medical device into a lumen of a patient. The medical device may include a porous body coupled to a distal end of a conduit, a proximal loop extending proximally from the porous body, and a distal loop extending distally from the porous body. The method may further include using the distal loop to move the medical device into a wound cavity. Upon using the distal loop to move the medical device into the wound cavity, the distal loop may be on a first side of a wall defining the lumen. The porous body may extend through at least a portion of the wall defining the lumen. The proximal loop may be on a second side of the wall defining the lumen. The method may further include applying a vacuum pressure to the porous body via the conduit and pulling the proximal loop in a proximal direction to remove the medical device from the wound cavity.

In some aspects, upon pulling the proximal loop in the proximal direction, a length of the porous body may increase, and a width of the porous body may decrease.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a perspective view of a distal portion of a medical system, according to aspects of this disclosure.

FIG. 2 is a flow diagram of an exemplary method, according to aspects of this disclosure.

FIGS. 3A and 3B illustrate side views of a distal portion of an exemplary endoluminal device in a first configuration (FIG. 3A) and a second configuration (FIG. 3B), according to aspects of this disclosure.

FIG. 4 illustrates a side view of a distal portion of another alternative endoluminal device, according to aspects of this disclosure.

FIG. 5 illustrates a side view of a distal portion of yet another alternative endoluminal device, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when placing the device into the subject. Proximal and distal directions are labeled with arrows marked “P” and “D,” respectively, throughout various figures. The terms and definitions provided herein control, if in conflict with terms and/or definitions incorporated by reference.

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of +10% in a stated value or characteristic. Additionally, terms that indicate the geometric shape of a component/surface encompass both exact and approximate shapes.

Although a target site is discussed herein as being in a subject's gastrointestinal tract, this disclosure is not so limited, as the target site may be any internal lumen, organ, cavity, or other tissue within the subject. Additionally, although endoscopes are referenced herein, it will be appreciated that the disclosure encompasses various devices that may be inserted into a body of a subject, such as ureteroscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

Endoluminal vacuum therapy (EVT or EVAC, and referred to herein as EVAC) is a procedure to treat wounds. For example, EVAC may be used to treat post-surgical leaks or perforations in the gastrointestinal tract (GI) following a surgical or endoscopic procedure, such as colonic resection, bariatric surgery, or esophagectomy. In EVAC, negative pressure is delivered to the wound site in the GI tract, for example, through a porous body such as, for example, a sponge/porous body (e.g., a vacuum-sealed porous body) at a distal end of a nasogastric tube. A proximal end of the nasogastric tube may be connected to a collection container. The sponge is placed endoscopically into the perforation, leak, or other wound.

In some examples, EVAC includes endoluminal placement of a sponge or other like material into the wound (e.g., target) site, including a perforation, a leak, a cyst, an anastomosis, etc. Placement of the material may be via a catheter, scope (endoscope, bronchoscope, colonoscope, duodenoscope, gastroscope, etc.), tube, or sheath, inserted into the GI tract via an incision or natural orifice. The natural orifice can be, for example, the nose, mouth, or anus, and the placement can be in any portion of the GI tract, including the esophagus, stomach, duodenum, large intestine, or small intestine. In some examples, the placement may be in or through a wall of the GI tract. Placement of the material can also be in other organs reachable via the GI tract (e.g., the colon). Negative pressure is then applied through the tube or porous body.

Applying the negative pressure through the porous body in the wound may help accelerate healing, for example, by encouraging local tissue granulation at a wound site. The porous body may be replaced with increasingly smaller sizes of porous bodies, for example, as the wound heals and/or closes. Devices and systems suited for EVAC are limited. During EVAC, a porous body may be delivered to a target site using a scope, rat-tooth forceps, and a suture loop at a distal end of the porous body. For example, the rat-tooth forceps may be extended through a working channel of the scope and used to grasp the loop at the distal end of the porous body. The rat-tooth forceps is used to move the porous body to a target site as the scope is navigated to the target site, which can present challenges. For example, the porous body may block or impair scope visualization and/or maneuverability as a user moves the porous body through the GI tract using the rat-tooth forceps.

Removal of a porous body from a target site during EVAC also presents challenges. For example, tissue may grow into the porous body if the porous body is left at the target site for an extended period of time. The tissue growth may make it difficult to remove the porous body from the target site, e.g., during porous body replacement. As such, EVAC typically requires a porous body to be replaced every 3 to 5 days to prevent ingrowth and to maintain cleanliness of the target site.

Aspects of this disclosure include systems, devices, and methods to help improve delivery and/or removal of an implant (e.g., a porous body) to a target site during an EVAC procedure. For example, aspects of this disclosure include systems, devices, and methods to help reduce the time needed to deliver an implant (e.g., a porous body) to a target site and/or reduce the time needed to remove an implant from a target site. Aspects of this disclosure may improve implant (e.g., porous body) exchanges at a target site.

Components of the systems and/or devices described herein may be packaged as a kit for EVAC. For example, the devices disclosed herein may include a vacuum tube and/or a vacuum source. Additionally, a connector coupled to a proximal portion of a porous body may be configured to couple a distal end or distal portion of the vacuum tube with the porous body to treat a wound. The vacuum tube may be coupled to a vacuum source, for example, to apply negative pressure to tissue (e.g., via the porous body) of the target site.

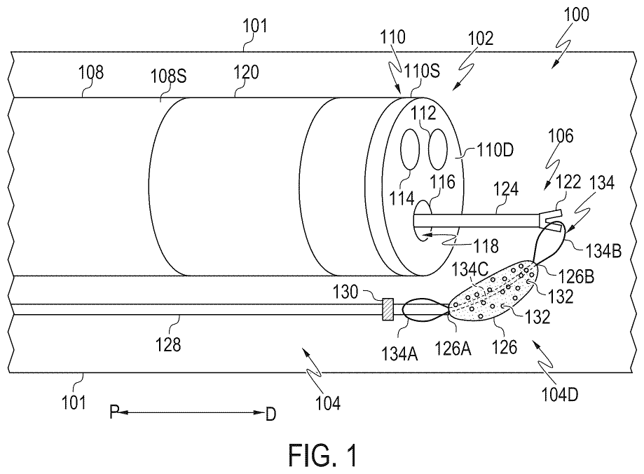

FIG. 1 illustrates a distal portion of a medical system 100. Medical system 100 may be inserted into a lumen 101 of a subject. Medical system 100 may be configured to treat wounds and/or to otherwise treat the subject. Medical system 100 may include a delivery device 102, a first medical device 104, and a second medical device 106. As will be discussed in further detail below, first medical device 104 may be an exemplary EVAC device that may be inserted into the subject, e.g., for treatment of a wound and/or to otherwise assist in treating subject's tissue. Delivery device 102 and/or second medical device 106 may be configured to assist in positioning and/or repositioning first medical device 104 within lumen 101 and/or within the tissue of the subject. Although not shown, medical system 100 may include one or more additional medical components (e.g., additional delivery devices and/or additional accessory instruments), for example, to assist in treating the subject.

In some examples, delivery device 102 may be a scope (e.g., an endoscope, a gastroscope, a duodenoscope, etc.). Delivery device 102 may include an insertion portion 108 (e.g., a shaft or a catheter) terminating at a distal tip 110. Distal tip 110 of delivery device 102 may include one or more imaging devices 112 (e.g., cameras, image sensors, lenses, etc.) and/or lighting elements 114 (e.g., light emitting diodes, or LEDs, optical fibers, etc.). For example, lighting element(s) 114 may be used (e.g., in combination with imaging device 112) to enable a user to visualize and navigate within lumen 101, e.g., without the need for invasive surgery. In some examples, imaging device(s) 112 and/or lighting elements 114 may be disposed on a distal face 110D of distal tip 110 (i.e., for a “forward-facing” device). In other examples, imaging device(s) 112 and/or lighting element(s) 114 may be disposed on a surface 110S of distal tip 110 and/or a side surface 108S of insertion portion 108 (i.e., for a “side-facing” device).

Additionally or alternatively, distal face 110D of distal tip 110 may include one or more distal openings. For example, distal face 110D may include an opening 116 of a working channel 118. Working channel 118 may extend (e.g., longitudinally) through insertion portion 108. Working channel 118 of delivery device 102 may be configured to receive second medical device 106. Although not shown, distal tip 110 may include additional openings, e.g., configured to provide irrigation, insufflation, and/or suction at distal tip 110.

Insertion portion 108 may further include an articulation portion 120. Articulation portion 120 may be configured to bend distal tip 110 in one or more directions (e.g., left, right, up, and/or down). Although not shown, delivery device 102 may include a handle coupled to a proximal end of insertion portion 108. The handle may include one or more ports (e.g., to receive second medical device 106) connected to working channel 118. Additionally or alternatively, the handle may include one or more actuators (e.g., buttons, knobs, levers, locks, etc.). The one or more actuators (not shown) may be configured to control one or more aspects of articulation portion 120 and/or distal tip 110. For example, the one or more actuators may be configured to control movement of articulation portion 120, for example, via one or more steering, or articulation, wires (not shown) that extend longitudinally through insertion portion 108 and to distal tip 110. Additionally or alternatively, one or more additional actuators (e.g., buttons, knobs, levers, etc.) may be configured to capture an image and/or video, and/or provide other functionality to distal tip 110.

As previously discussed, second medical device 106 may be movably disposed within working channel 118 of insertion portion 108. A distal end of second medical device 106 may include graspers, forceps, rat-tooth forceps, or any other medical device configured to grasp first medical device 104. For example, second medical device 106 may include a pair of movable jaws 122 coupled (e.g., directly or indirectly) to a distal end of a sheath 124 of second medical device 106. Although not shown, second medical device 106 may include a handle coupled to a proximal end of sheath 124. The handle (not shown) may be operably connected to one or more control wires or other elements such that actuation of one or more aspects of the handle is configured to open/close jaws 122 and/or to otherwise articulate a distal portion of second medical device 106.

First medical device 104 may be external to delivery device 102. For example, first medical device 104 may extend alongside or adjacent to delivery device 102. A distal end 104D of first medical device 104 may include a porous body 126 (e.g., an implant), such as a sponge or other porous body. First medical device 104 may also include a tube or conduit 128, and porous body 126 may be coupled (e.g., directly or indirectly) to a distal end of conduit 128. In some examples, first medical device 104 may further include a coupler 130. Coupler 130 may be configured, for example, to couple, or connect, a distal end of conduit 128 to a proximal end of porous body 126. Coupler 130 may include a quick-connect fitting, a luer fitting, or any other fitting configured to couple porous body 126 with conduit 128 and permit fluid (e.g., liquid or gas) flow between porous body 126 and conduit 128. Although coupler 130 is illustrated as being disposed near, or adjacent to, distal end 104D of first medical device 104, coupler 130 may be disposed anywhere along conduit 128 (e.g., at or near a proximal portion of conduit, at or near a center portion of conduit, etc.).

Porous body 126 may have any features of any porous body that is known in the art for use in EVAC procedures. For example, porous body 126 may include an open-cell porous body. Porous body 126 may include a plurality of openings 132 (hereinafter referred to as “openings”) on an outer surface and/or interior thereof. Openings 132 may be any hole, pore, or channel. Openings 132 may include interconnecting channels and/or pores throughout porous body 126. For example, openings 132 may be pores of porous body 126. Openings 132 may have different sizes and/or shapes. Features of openings 132 (e.g., a size and shape of pores of porous body 126) may be selected based on a location of treatment within the body, properties of a wound to be treated, a stage of treatment, or one or more other factors. Porous body 126 is illustrated as having a cylindrical shape with a tapered proximal end 126A and a tapered distal end 126B, but may be any shape, including spherical, cuboidal, irregular, or the like. Porous body 126 may be compressed into a lower profile or smaller size during insertion to a target site, and porous body 126 may expand during deployment at a target site.

Porous body 126 may include any suitable biocompatible material that may absorb liquids and/or permit liquid or other materials to pass therethrough via, e.g., negative pressure applied to porous body 126. The material of porous body 126 may be flexible, compressible, porous, hydrophilic, sterile, and/or disposable. Suitable materials include polyurethanes, esters, ethers, composite materials, and/or other medical-grade materials. Additionally or alternatively, porous body 126 may include (e.g., be doped with, treated with, infused with, etc.) one or more medications, for example, to assist in promoting tissue growth, preventing infection, assist with pain management, etc.

Distal end 104D of first medical device 104 may further include a proximal loop 134A and a distal loop 134B. Proximal loop 134A and distal loop 134B may be formed from an elongate body 134. For example, proximal loop 134A may extend proximally, or radially outward, from proximal end 126A of porous body 126. Distal loop 134B may extend distally from distal end 126B of porous body 126. In some examples, each of proximal loop 134A and distal loop 134B may be individual loops formed from two or more elongate bodies 134. For example, proximal loop 134A may be formed from a first elongate body and/or distal loop 134B may be formed from a second elongate body. Alternatively, a center portion 134C of elongate body 134 may extend between proximal loop 134A and distal loop 134B, thereby coupling, or connecting, proximal loop 134A and distal loop 134B. Center portion 134C may extend longitudinally (e.g., proximally or distally) through a center portion of porous body 126, as illustrated in FIG. 1 by the dashed lines. Although not shown, in further alternative examples, proximal loop 134A and distal loop 134B may be portions of a larger, single loop. In such an example, the middle portion of the larger loop (e.g., the portion extending between proximal loop 134A and distal loop 134B) may extend through, or within, porous body 136.

Elongate body 134 (e.g., each of proximal loop 134A, distal loop 134B, and center portion 134C) may include, for example, one or more threads, wires, and/or cables formed from any number of materials or combinations of materials. For example, one or more portions (e.g., proximal loop 134A, distal loop 134B, and/or center portion 134C) of elongate body 134 may be comprised of one or more threads (e.g., suture threads). For example, the one or more threads comprising elongate body 134 may be surgical steel, silk, cotton, linen, nylon, polypropylene, and/or polybutester thread(s). Additionally or alternatively, one or more portions of elongate body 134 may be comprised of one or more wires or cables. The wire(s) and/or cable(s) may be comprised of stainless steel, platinum, titanium, cobalt-chrome alloys, nickel-titanium alloy, or other medical-grade metals. Additionally or alternatively, one or more portions of elongate body 134 may be comprised of one or more elastic materials, such as, for example, one or more elastomers (e.g., silicone rubber, latex, rubber latex), elastic polymers (e.g., polyurethane, thermoplastic elastomers, or TPE) and/or other medical-grade materials having elastic properties.

In some examples, an entirety of elongate body 134 (e.g., each of proximal loop 134A, distal loop 134B, and center portion 134C) may be formed from a same material. For example, elongate body 134 may be comprised of a stainless steel wire, a single suture, or a nickel-titanium cable. Alternatively, one or more portions of elongate body 134 may be comprised of different materials. For example, each of proximal loop 134A and distal loop 134B may be formed of a first material having flexible, bendable, and/or elastic characteristics, and center portion 134C may be formed from a second material having more rigid, inflexible, or inelastic properties. For example, proximal loop 134A may be comprised of a suture thread, distal loop 134B may be comprised of a nickel-titanium wire, and center portion 134C may be comprised of a stainless steel cable. In such an example, proximal loop 134A and/or distal loop 134B may be coupled, or connected to, center portion 134C portion via, for example, a crimp, a ferrule, a knot, an adhesive, an epoxy, or any other one or more coupling mechanisms commonly used in the art, for example, to couple two or more wires/cables/threads.

In some examples, each portion of elongate body 134 may be comprised of a same material and have the same physical properties. For example, each portion of elongate body 134 (e.g., proximal loop 134A, distal loop 134B, and center portion 134C) may be comprised of the same material having the same physical properties. Alternatively, one or more portions of elongate body 134 may be comprised of two or more different materials and yet still have the same physical properties. For example, a cross-sectional dimension and/or shape of the one or more wires/cable/threads forming elongate body 134 may be the same or similar for each of proximal loop 134A, distal loop 134B, and center portion 134C.

In alternative examples, one or more portions of elongate body 134 may be comprised of the same material, yet have different physical properties. In such an example, elongate body 134 may be comprised of a single material, and a cross-sectional dimension and/or cross-sectional shape of one or more portions of elongate body 134 may differ. For example, a cross-sectional dimension and/or a cross-sectional shape of proximal loop 134A and/or distal loop 134B may differ from a cross-sectional dimension and/or shape of center portion 134C.

Alternatively, one or more portions of elongate body 134 may be comprised of different materials and have different physical properties. For example, center portion 134C may have a greater cross-sectional dimension as compared to proximal loop 134A and/or distal loop 134B. In some examples, a greater cross-sectional dimension of center portion 134C may provide additional structural support to porous body 126. For example, center portion 134C may be configured to limit the ability for porous body 126 to bend or curve (e.g., adding structural support for porous body 126). In alternative examples, a cross-sectional dimension/shape of proximal loop 134A and/or distal loop 134B may be greater than center portion 134C, for example, to withstand proximal (e.g., pulling) forces and/or distal (e.g., pushing) forces exerted on proximal loop 134A and/or distal loop 134B by a user.

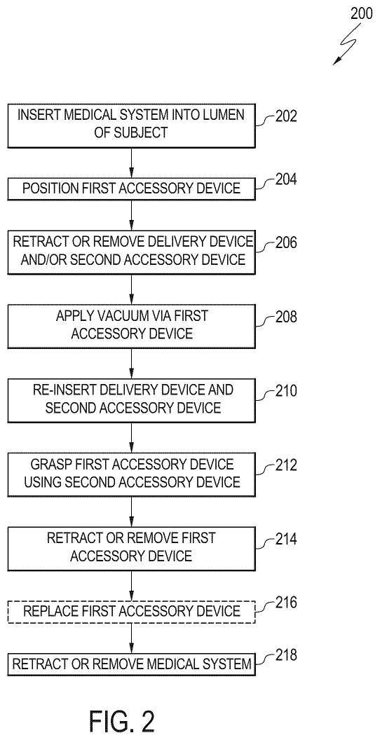

FIG. 2 illustrates an exemplary process, or method 200, that one or more users may perform with any of the medical systems, devices, or portions of systems discussed herein, for example, medical system 100. Referring to both FIGS. 1 and 2, in a step 202, medical system 100 may be inserted into lumen 101 of the subject and navigated to the target tissue. In some examples, all components of medical system 100 (e.g., delivery device 102, first medical device 104, and second medical device 106) may be inserted into lumen 101 together, or at once. In other examples, each component of medical system 100 may be inserted individually (e.g., one at a time). In such an example, delivery device 102 may first be inserted into lumen 101. First medical device 104 may then be inserted alongside delivery device 102 and/or second medical device 106 may be inserted through working channel 118 of delivery device 102. First medical device 104 and second medical device 106 may be inserted such that a distal end of each device is distal to distal face 110D of delivery device 102.

Once medical system 100 is in position, a step 204 includes positioning a first accessory device. For example, in step 204, first medical device 104 may be positioned using second medical device 106 and/or delivery device 102. For example, second medical device 106 may grasp at least a portion of distal loop 134B of first medical device 104 (e.g., via jaws 122). First medical device 104 and second medical device 106 may then be maneuvered (e.g., moved proximally or distally), for example, to position first medical device 104 as desired. For example, second medical device 106 may be moved proximally and/or distally within working channel 118 of delivery device 102 and/or delivery device 102 may be maneuvered with second medical device 106 so as to maneuver first medical device 104. Additionally or alternatively, articulation portion 120 of delivery device 102 may be articulated (e.g., bent), for example, to assist in maneuvering first medical device 104 and/or second medical device 106 to the desired position within lumen 101. Additionally or alternatively, the user may manipulate conduit 128 (e.g., proximally and/or distally) to position porous body 126 to the desired position within lumen 101.

Although not shown, in some methods of use, first medical device 104 and second medical device 106 may be inserted into or through a wound. For example, first medical device 104 may be positioned such that distal loop 134B is on a first side (e.g., outside) of a wall defining lumen 101 and proximal loop 134A is on a second side (e.g., inside) of a wall defining lumen 101. As such, once first medical device 104 is in position, tissue may circumferentially surround porous body 126 of first medical device 104.

Next, a step 206 includes retracting to removing a delivery device and/or a second accessory device. For example, once first medical device 104 is in position, in step 206, delivery device 102 and/or second medical device 106 may be removed, or retracted proximally, from lumen 101. For example, jaws 122 of second medical device 106 may be opened (e.g., via the handle of second medical device 106) to release distal loop 134B. Second medical device 106 and/or delivery device 102 may be proximally removed from lumen 101 with first medical device 104 remaining inside the body. First medical device 104 may remain in lumen 101 for extended periods (e.g., several minutes to several days).

A step 208 includes applying vacuum or negative pressure via the first accessory device. For example, in step 208, suction, or a vacuum pressure, may be applied, for example, via porous body 126. In some aspects, porous body 126 may be positioned adjacent to tissue, so that the suction is applied to the tissue. The vacuum may be used to assist in removing excess fluid and/or promoting tissue growth, e.g., into porous body 126.

After a period of time, the first accessory device (i.e., first medical device 104) may need to be removed or repositioned. For example, a step 210 includes reinserting the delivery device and the second accessory device. In these aspects, step 210 may include reinserting delivery device 102 and/or second medical device 106 into lumen 101 and maneuvering to the target site. In some examples, delivery device 102 may be inserted into lumen 101 first, and then second medical device 106 may be inserted through working channel 118 of delivery device 102.

Additionally, a step 212 includes grasping the first accessory device using the second accessory device. For example, in step 212, second medical device 106 may be used to grasp proximal loop 134A of first medical device 104 (e.g., via jaws 122). For example, jaws 122 of first medical device 104 may be opened and closed (e.g., via the handle of second medical device 106) so as to grasp proximal loop 134A between jaws 122 of second medical device 106.

Next, a step 214 includes retracting or removing the first accessory device. For example, in step 214, second medical device 106 and/or delivery device 102 may be pulled proximally (e.g., retracted), for example, to release, or remove first medical device 104 from the tissue or the target site. First medical device 104 may then be removed (e.g., pulled proximally) from lumen 101, thus removing porous body 136 from lumen 101. In other examples, porous body 136 may be decoupled or disconnected from conduit 128 (e.g., via coupler 130). As such, conduit 128 may separately be retracted from lumen 101. With conduit 128 decoupled from porous body 136, porous body 136 may be removed via working channel 118 of delivery device 102. For example, second medical device 106 may grasp porous body 136, proximal loop 134A, and/or distal loop 134B (e.g., using jaws 122) and porous body 136 may be pulled proximally or retracted through working channel 118 of delivery device 102.

An optional step 216 includes replacing the first accessory device, for example, with another first accessory device. In these aspects, with first medical device 104 removed from lumen 101, in optional step 216, a new first medical device 104 (e.g., a new porous body 126) may be inserted and positioned within the tissue, as described above. For example, as the wound or tissue heals, porous body 136 of first medical device 104 may be replaced periodically (e.g., with incrementally decreasing sizes). First medical device 104 may be replaced as many times as necessary, for example, until the tissue at the target site is sufficiently, or desirably, healed.

Lastly, a step 218 includes retracting or removing the medical system. For example, once first medical device 104 is removed (e.g., retracted) and/or replaced sufficiently, medical system 100 (e.g., delivery device, 102, first medical device 104, and second medical device 106) may be retracted or removed from the subject in step 218. Various aspects of method 200 (e.g., one or more of steps 202-218) may be repeated as many times as desired.

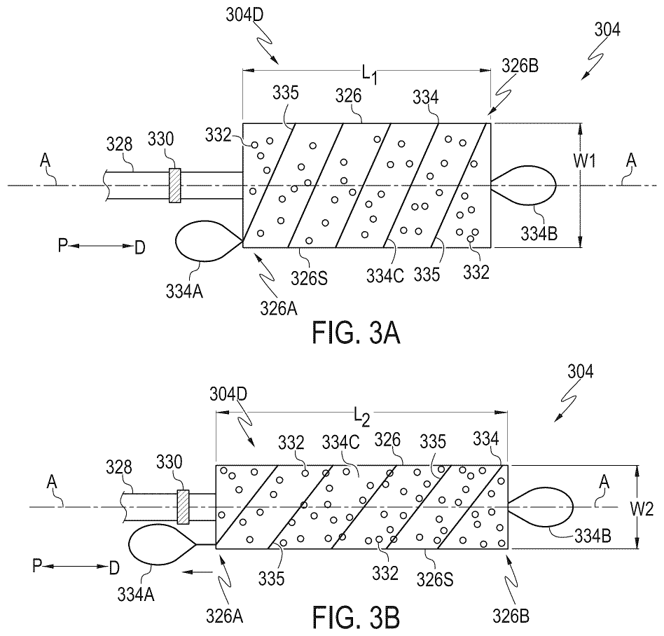

FIGS. 3A and 3B illustrate an alternative embodiment of a first medical device 304 in a first configuration (FIG. 3A) and in a second configuration (FIG. 3B). For example, first medical device 304 may be an EVAC device. First medical device 304 may be used with one or more aspects of medical system 100. Additionally, method 200 may be performed using first medical device 304 (e.g., in conjunction with delivery device 102 and/or second medical device 106).

Except as described below, first medical device 304 may have any or all of the characteristics of first medical device 104, as described above with respect to FIG. 1. For example, a distal end 304D of first medical device 304 may include a porous body 326 removably or fixedly coupled (e.g., directly or indirectly) to a distal end of a conduit 328. Porous body 326 may have any or all of the characteristics of porous body 126, described above. For example, porous body 326 may include openings 332. Openings 332 may be any hole, pore, or channel. Openings 332 may include interconnecting channels and/or pores throughout porous body 326. Openings 332 may be pores of porous body 326. Additionally, conduit 328 may have any or all of the characteristics of conduit 128, described above. For example, conduit 328 may include a coupler 330. Coupler 330 may be configured, for example, to couple, or connect, conduit 328 to porous body 326.

First medical device 304 may further include a proximal loop 334A and a distal loop 334B formed from an elongate body 334. For example, proximal loop 334A may extend proximally, or radially outward, from a proximal end 326A of porous body 326. In some examples, proximal loop 334A may extend proximally from side of porous body 326. For example, proximal loop 334A may be offset from an axis A extending longitudinally through a center of porous body 326 and conduit 328. Distal loop 334B may extend distally from distal end a 326B of porous body 326. In some examples, distal loop 334B may be aligned with axis A. A center portion 334C of elongate body 134 may extend between proximal loop 334A and distal loop 334B. Each portion of elongate body 334 (e.g., proximal loop 334A, distal loop 334B, and center portion 334C) may have any or all of the characteristics of the respective portions of elongate body 134, except as described below.

In some examples, a distal end of center portion 334C may be fixed to porous body 326 (e.g., via a knot, a splice, a crimp, a fastener, etc.). Additionally or alternatively, the distal end of center portion 334C may be coupled to distal loop 334B (e.g., via a knot, a splice, a crimp, a fastener, etc.). In some examples, a proximal end of center portion 334C may be coupled to proximal loop 334A (e.g., via a knot, a splice, a crimp, a fastener, etc.). In some examples, the knot, splice, crimp, fastener, etc. between proximal loop 334A and center portion 334C and/or between distal loop 334B and center portion 334C may be configured to anchor each respective portion of elongate body 334 to or within porous body 326. As such, the knot, splice, crimp, fastener, etc. may assist in preventing each of proximal loop 334A, distal loop 334B, and/or center portion 334C from moving relative to one another and/or relative to porous body 326.

Center portion 334C may include a helical structure surrounding at least a portion of porous body 326. For example, center portion 334C may include a plurality of turns 335 that extend circumferentially around porous body 326. In some examples, center portion 334C may extend circumferentially around an outer surface 326S of porous body 326. In other examples, center portion 334C may be at least partially embedded within porous body 326 (e.g., the plurality of turns 335 may extend at least partially into or through respective portions of porous body 326).

In a first configuration, as shown in FIG. 3A, porous body 326 may have a first length L₁, and a first width W₁. FIG. 3A may be illustrative of the first configuration of first medical device 304, for example, before, during, and/or after placement of first medical device 304 into the subject (e.g., within the target tissue of the subject). During delivery or implantation (e.g., while porous body 326 is in the target tissue), tissue may grow into porous body 326, and thus, in some configurations, anchor at least some one or more portions of porous body 326 into the tissue of the subject.

In a second configuration, as shown in FIG. 3B, porous body 326 may be elongated and compressed, for example, during removal of first medical device 304 from the target tissue. For example, in the second configuration porous body 326 may have a second length L₂ and a second width W₂. Second length L₂ may be greater, or longer, than first length L₁. Second width W₂ may be less, or smaller, than first width W₁. Porous body 326 may be elongated by, for example, pulling proximal loop 334A proximally. For example, although not shown, a second medical device (e.g., second medical device 106 of FIG. 1) may be used to grasp and pull proximal loop 334A in a proximal direction to help transition porous body 326 from the first configuration (FIG. 3A) to the second configuration (FIG. 3B).

As proximal loop 334A is being pulled proximally, center portion 334C may help to constrict, or compress, porous body 326, thus helping to decrease the width of porous body 326 and increasing an overall length of porous body 326. Center portion 334C constricts, or compresses porous body 326, for example, by nature of the plurality of turns 335 of center portion 334C. In some examples, center portion 334C may be configured to uniformly compress, or constrict porous body 326.

In other examples, porous body 326 may be compressed, for example, as proximal loop 334A is pulled proximally. As proximal loop 334A is pulled proximally, a diameter of each turn of the plurality of turns 335 of center portion 334C may decrease. Thus, as proximal loop 334A is pulled proximally, a spacing between adjacent turns of the plurality of turns 335 may increase. In some aspects, for example, as the space between adjacent turns of the plurality of turns 335 increases, the diameter or width of porous body 326 may decrease. Accordingly, porous body 326 is constricted, or compressed.

In other aspects, the spaces between adjacent turns of the plurality of turns 335 may not change (e.g., increase or decrease). For example, the knot, crimp, splice, fastener, etc. that may be formed between proximal loop 334A and center portion 334C and/or between distal loop 334B and center portion 334C may also be fixed to porous body 326. As such, the knot, crimp, splice, fastener, etc. may be configured to prevent the spaces between adjacent turns of the plurality of turns 335 from increasing or decreasing. Furthermore, in some examples, center portion 334C may include a plurality of anchors dispersed along a length of center portion 334C. For example, the plurality of anchors (e.g., knots, crimps, fasteners, etc.) may be configured to further anchor center portion 334C to or within center portion 334C. In some aspects, the plurality of anchors may be configured to prevent a change in the amount of space between adjacent turns of the plurality of turns 335

Compressing, or constricting, porous body 326 may assist in dislodging porous body 126 from any tissue that has grown into or through porous body 326. For example, compressing porous body 326 may result in porous body 326 being unanchored from the tissue. Thus, once porous body 326 has been unanchored, or dislodged, from the tissue, an entirety of first medical device 304 may be removed from the subject and/or replaced. Furthermore, in some examples, porous body 326 may be movable over at least a portion of conduit 328 (e.g., as porous body 326 is compressed). As such, a distance between coupler 330 and a proximal end of porous body 326 may be reduced between the first configuration (FIG. 3A) and the second configuration (FIG. 3B).

As proximal loop 334A is pulled proximally, porous body 326 may bend or bow, e.g., based on the relation of proximal loop 334A being offset from axis A. The helical structure of center portion 334C may be configured to assist in reducing the degree of which porous body 326 may bend or bow, for example, by nature of the uniform compressive/constrictive forces being applied to the porous body by center portion 334C, for example, as proximal loop 334A is pulled proximally.

Additionally or alternatively, one or more support structures (not shown) may be disposed within porous body 326. For example, one or more support structures may extend longitudinally (e.g., proximally and distally) within porous body 326. The support structures may be comprised of a stiff (e.g., rigid, or semi-rigid) material, for example, to help prevent porous body 326 from bending/bowing during use. For example, the support structures (not shown) may be comprised one or more plastics and/or metals. As such, the support structures may help prevent bending/bowing of porous body 326, for example, during the positioning of porous body 326 within the subject and/or removal of porous body 326 from the subject.

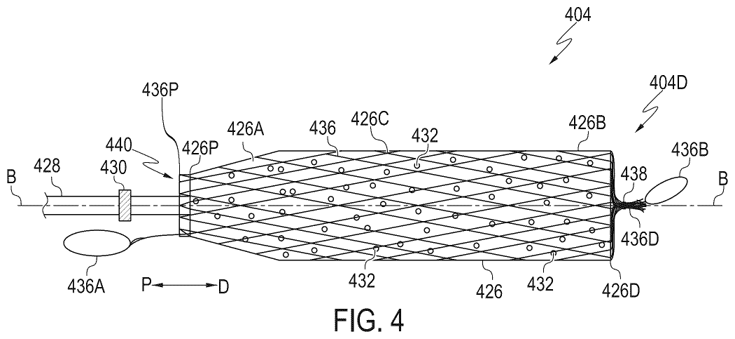

FIG. 4 illustrates an alternative embodiment of a first medical device 404. Except as described below, first medical device 404 may have any or all of the characteristics of first medical device 104 of FIG. 1 and/or first medical device 304 of FIGS. 3A and 3B. For example, a distal end 404D of first medical device 404 may include a porous body 426 removably or fixedly coupled (e.g., directly or indirectly) to a distal end of a conduit 428. Additionally, method 200 may be performed using first medical device 404 (e.g., in conjunction with delivery device 102 and/or second medical device 106 of FIG. 1).

Porous body 426 of first medical device 404 may have any or all of the characteristics of porous body 126 and/or porous body 326, described above. For example, porous body 426 may include openings 432. Openings 432 may be any hole, pore, or channel. Openings 432 may include interconnecting channels and/or pores throughout porous body 426. Openings 432 may be pores of porous body 426. Additionally, conduit 428 may have any or all of the characteristics of conduit 128 and/or conduit 328, described above. For example, conduit 428 may include a coupler 430.

Porous body 426 is comprised of a proximal portion 426A and a distal portion 426B. A center portion 426C may extend between proximal portion 426A and distal portion 426B. Proximal portion 426A of porous body 426 may taper (e.g., towards a proximal end 426P of porous body 426). For example, proximal end 426P of porous body 426 may have a smaller diameter, e.g., as compared to distal portion 426B, a distal end 426D of porous body 426, and/or center portion 426C of porous body 426. Although not shown, distal portion 426B of porous body 426 may be similarly tapered. For example, distal portion 426B may taper towards distal end 426D of porous body 426 such that distal end 426D of porous body 426 has a smaller diameter (e.g., as compared to center portion 426C).

First medical device 404 may further include a netting 436 at least partially surrounding, or encompassing, porous body 426. For example, netting 436 may extend circumferentially around an outer surface of porous body 426 (e.g., from proximal end 426P of porous body 426 to distal end 426D of porous body 426). In other examples, netting 436 may be at least partially embedded within porous body 426 (e.g., netting 436 may extend at least partially into or through respective portions of porous body 426.

Netting 436 may be comprised of a mesh, a webbing, a lattice, and/or a network of one or more threads, wires, and/or cables. For example, netting 436 may be comprised of surgical steel, silk, cotton, linen, nylon, polypropylene, and/or polybutester thread(s). Additionally or alternatively, netting 436 may be comprised of one or more wires or cables. The wire(s) and/or cable(s) may be comprised of stainless steel, platinum, titanium, cobalt-chrome alloys, nickel-titanium alloy, or other medical-grade metals. Additionally or alternatively, netting 436 may be comprised of one or more elastic materials, such as, for example, elastomers (e.g., silicone rubber, latex, rubber latex), elastic polymers (e.g., polyurethane, thermoplastic elastomers, or TPE) and/or other medical-grade materials having elastic properties.

Netting 436 may be configured conform to a size and/or shape of porous body 426, for example, without exerting compressive forces to porous body 426. In other examples, netting 436 may be configured to exert compressive, or radially-inward, forces, for example, to shape and/or otherwise form porous body 426. For example, netting 436 may be configured to form the tapered portions of porous body 426 (e.g., proximal portion 426A and/or distal portion 426B of porous body 426).

In some examples, a proximal end 436P of netting 436 may extend proximally past proximal end 426P of porous body 426. Additionally or alternatively, a distal end 436D of netting 436 may extend distally past distal end 426D of porous body 426. In some examples, distal end 436D of netting 436 may be closed, or cinched, for example, via a fastener 438. Fastener 438 may include a crimp, a ferrule, a knot, a bead of adhesive or epoxy, and/or any other mechanism(s) for closing or cinching a distal end 436D of netting 436. Although not shown, proximal end 436P of netting 436 may be similarly closed or cinched together. In other examples, proximal end 426P may include an opening 440. Opening 440 may be oriented perpendicular to an axis A extending longitudinally (e.g., proximally to distally) through first medical device 404.

Netting 436 may include a proximal loop 436A extending proximally from a proximal end 436P of netting 436. Netting 436 may further include a distal loop 436B extending distally from a distal end 436D of netting 436. Proximal loop 436A and/or distal loop 436B may be formed from one or more wires, cables, and/or threads. In some examples, each of proximal loop 436A and/or distal loop 436B may be integrally formed with netting 436. Alternatively, proximal loop 436A and/or distal loop 436B may be coupled (e.g., connected to), netting 436 via, for example, a crimp, a ferrule, a knot, an adhesive, an epoxy, and/or any other means or combination of means commonly used in the art, for example, to couple two or more wires/cables/threads.

In some examples, proximal loop 436A may extend proximally from proximal end 426P of porous body 426. For example, proximal loop 436A may be offset from axis B. Distal loop 436B may extend distally past a distal end 426D of porous body 426. In some examples, distal loop 436B may extend from distal end 404D of first medical device 404. For example, distal loop 436B may be aligned with axis B.

First medical device 404 may be configured such that, for example, as proximal loop 436A is pulled proximally, netting 436 may constrict, or compress, at least a portion of porous body 426. In some examples, netting 436 may uniformly constrict, or compress, porous body 426. For example, as proximal loop 436A is pulled proximally, a cross-sectional diameter of porous body 426 may decrease. In some examples, a length of porous body 426 may increase, for example, as proximal loop 436A is pulled proximally. Constricting, or compressing, porous body 426 may assist in dislodging porous body 426 from any tissue that has grown into or through porous body 426. For example, compressing porous body 426 may result in porous body 426 being unanchored from the tissue. Thus, once porous body 426 has been unanchored, or dislodged, from the tissue, an entirety of first medical device 404 may be removed from the subject and/or replaced.

As proximal loop 436A is pulled proximally, porous body 426 may bend or bow, e.g., based on the relation of proximal loop 436A being offset from axis B. Netting 436 may be configured to assist in reducing the degree of which porous body 426 may bend or bow for example, by nature of the uniform compressive/constrictive forces being applied to the porous body. For example, as proximal loop 436A is pulled proximally, netting 436 may assist in reducing the degree of which porous body 426 bens or bows.

Additionally or alternatively, one or more support structures (not shown) may be disposed within porous body 426. For example, one or more support structures may extend longitudinally (e.g., proximally and distally) within porous body 426. The support structures may be comprised of a stiff (e.g., rigid, or semi-rigid) material, for example, to help prevent porous body 426 from bending/bowing during use. For example, the support structures (not shown) may be comprised one or more plastics and/or metals. As such, the support structures may help prevent bending/bowing of porous body 426, for example, during the positioning of porous body 426 within the subject and/or removal of porous body 426 from the subject.

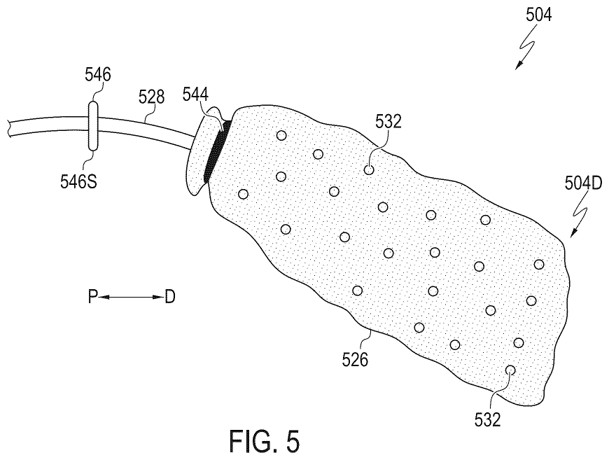

FIG. 5 illustrates another alternative embodiment of a first medical device 504. Except as described below, first medical device 504 may include any or all of the characteristics of first medical device 104 of FIG. 1, first medical device 304 of FIGS. 3A and 3B, and/or first medical device 404 of FIG. 4. For example, a distal end 504D of first medical device 504 may include a porous body 526 removably or fixedly coupled (e.g., directly or indirectly) to a distal end of a conduit 528. Additionally, method 200 may be performed using first medical device 504 (e.g., in conjunction with delivery device 102 and/or second medical device 106 of FIG. 1).

Porous body 526 of first medical device 504 may have any or all of the characteristics of porous body 126, porous body 326, and/or porous body 426, described above. For example, porous body 526 may include openings 532. Openings 532 may be any hole, pore, or channel. Openings 532 may include interconnecting channels and/or pores throughout porous body 526. Openings 532 may be pores of porous body 526. Additionally, conduit 528 may have any or all of the characteristics of conduit 128, conduit 328, and/or conduit 428, described above.

In some examples, a proximal portion of porous body 526 may include a fastener 544, for example, to assist in coupling porous body 526 to conduit 428. Fastener 544 may include, for example, a crimp, a ferrule, a knot, etc. surrounding an outer surface of porous body 526. In some examples, fastener 544 may form a cinched portion of porous body 526

First medical device 504 may further include a grip 546. Grip 546 may be disposed proximal of a proximal end of porous body 526, for example, along a distal portion of conduit 528. In some examples, grip 546 may be an O-ring, for example, to serve or provide a grasping point for second medical device 106. For example, grip 546 may be configured such that jaws 122 may grasp onto grip 546. As such, second medical device 106 may use grip 546, for example, to position/reposition first medical device 504 within the subject and/or to remove first medical device 504 from the subject. Furthermore, with grip 546 separate from and proximal to porous body 526, porous body 526 may be cut or otherwise sized/shaped by the user. For example, a distal portion of porous body 526 may be cut or otherwise removed in order to resize porous body 526, for example, based on a size of a wound in the lumen or at the target site.

In some examples, grip 546 may be integrally formed with conduit 528. In other examples, grip 546 may be a separate component. In such an example, grip 546 may be movably disposed along conduit 528. Alternatively, grip 546 may be a component fixed to conduit 528, for example, via an adhesive, an epoxy, a crimp, a ferrule, a swage, or any other coupling mechanisms commonly known in the art to fix two or more components together.

Grip 546 may be comprised of one or more biocompatible materials. For example, grip 546 may be comprised of an elastic material (e.g., a rubber, a rubber latex, etc.), a hard or soft plastic (e.g., polypropylene, polyethylene, polyvinyl chloride, etc.), a metal (e.g., stainless steel, titanium, etc.), and/or any other biocompatible material. Furthermore, although one grip 546 is illustrated in FIG. 5, first medical device 504 may include additional grips. In some examples, a first of grip 546 may be fixed to conduit 528, and a second of grip 546 may be movable along conduit 528.

A cross-section of grip 546 may ring-like, or torus shaped. For example, although not shown, grip 546 may have an opening configured to receive conduit 528. The opening may extend through a center portion of grip 546. An outer surface 546S of grip 546 may be smooth. Alternatively, outer surface 542S of grip 546 may include one or more features (e.g., extensions and/or indentations) extending radially outward from outer surface 546S of grip 546. The one or more features may be configured, for example, to provide additional portions for second medical device 106 to grasp or grip, for example, during the placement and/or removal of first medical device 504 within the subject. In some examples, a cross section of grip 546 may be star-shaped (e.g., with a plurality of pointed extensions/indentations), flower shaped (e.g., with a plurality of curved extensions/indentations), or any other shape, for example, to help to allow for second medical device 106 to grasp onto grip 546. Additionally or alternatively, grip 546 may be shaped similar to a figure “8,” (e.g., with at least two openings) In such an example, a first opening of grip 546 may be configured to receive conduit 528 and a second opening of grip 546 may be configured to receive second medical device 106. Alternatively, the second opening of grip 546 may be configured similar to a handle such that second medical device 106 may grasp grip 546 (e.g., using jaws 122).

As such, grip 546 and/or any features extending radially outward or proximally from grip 546 may be gripped using second medical device 106 (e.g., by jaws 122). Second medical device 106 and/or delivery device 102 may then be pulled proximally, thus pulling grip 546 and first medical device 104 proximally along with second medical device 106. As such, first medical device 504 may be removed from the subject via grip 546.

Although not shown, any or all of first medical device 104, first medical device 304, and/or first medical device 404 may include grip 546. For example, grip 546 may be coupled to conduit 128 of first medical device 104. Similarly, grip 546 may be coupled to conduit 328 of first medical device 304. Grip 546 may also be coupled to conduit 428 of first medical device 404.

It will be apparent to those skilled in the art that various modifications and variations can be made to the disclosed systems, devices, and methods without departing from the scope of the disclosure. In some examples, various components discussed herein may be used interchangeably with each embodiment. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A medical device, comprising:
a conduit;
a porous body coupled to a distal end of the conduit;
a first loop extending proximally from a proximal end of the porous body; and
a second loop extending distally from a distal end of the porous body.

2. The medical device of claim 1, wherein in a first configuration, the porous body has a first length and a first width, wherein in a second configuration, the porous body has a second length and a second width, wherein the second length is greater than the first length, and wherein the second width is less than the first width.

3. The medical device of claim 2, wherein, to transition the porous body from the first configuration to the second configuration, the first loop is pulled proximally.

4. The medical device of claim 1, wherein the first loop defines a proximal end of an elongate body, wherein the second loop defines a distal end of the elongate body, and wherein a center portion extends between each of the first loop and the second loop.

5. The medical device of claim 4, wherein each of the first loop and the second loop is integrally formed with the center portion.

6. The medical device of claim 4, wherein the center portion extends through at least a portion of the porous body.

7. The medical device of claim 4, wherein the center portion includes a plurality of turns.

8. The medical device of claim 7, wherein the plurality of turns extend circumferentially around at least a portion of the porous body.

9. The medical device of claim 1, wherein a netting extends between the first loop and the second loop, wherein the netting extends circumferentially around at least a portion of the porous body.

10. The medical device of claim 1, wherein a longitudinal axis extends through the conduit and the porous body.

11. The medical device of claim 10, wherein the second loop is offset from the longitudinal axis.

12. The medical device of claim 1, wherein at least one of the first loop or the second loop is comprised of at least one of a thread, a wire, or a cable.

13. The medical device of claim 1, wherein at least one of the first loop or the second loop is a suture.

14. The medical device of claim 1, wherein the conduit includes a coupler, wherein the coupler is configured to removably couple the conduit and the porous body.

15. The medical device of claim 1, wherein the conduit is configured to be coupled to a vacuum source, wherein the vacuum source is configured to provide a negative pressure to the porous body.

16. A medical system, comprising:
a delivery device including a working channel extending therethrough;
a first medical device, wherein the first medical device is movably disposed within the working channel of the delivery device; and
a second medical device, wherein the second medical device comprises:
a conduit,
a porous body coupled to a distal end of the conduit, and
an elongate body, wherein a proximal end of the elongate body includes a first loop, wherein a distal end of the elongate body includes a second loop.

17. The medical system of claim 16, wherein a center portion of the elongate body includes a plurality of turns, wherein the plurality of turns extend circumferentially around at least a portion of the porous body,
wherein in a first configuration, the porous body has a first length and a first width, wherein in a second configuration, the porous body has a second length and a second width, wherein the second length is greater than the first length, wherein the second width is less than the first width, and
wherein, to transition the porous body from the first configuration to the second configuration, the first loop is pulled proximally.

18. The medical system of claim 16, wherein the second medical device is movably disposed adjacent to the delivery device.

19. A method for performing a medical procedure, the method comprising:
inserting a medical device into a lumen of a patient, the medical device including:
a porous body coupled to a distal end of a conduit,
a proximal loop extending proximally from the porous body, and
a distal loop extending distally from the porous body;
using the distal loop to move the medical device into a wound cavity, wherein, upon using the distal loop to move the medical device into the wound cavity, the distal loop is on a first side of a wall defining the lumen, the porous body extends through at least a portion of the wall defining the lumen, and the proximal loop is on a second side of the wall defining the lumen;
applying a vacuum pressure to the porous body via the conduit; and
pulling the proximal loop in a proximal direction to remove the medical device from the wound cavity.

20. The method of claim 19, wherein, upon pulling the proximal loop in the proximal direction, a length of the porous body increases and a width of the porous body decreases.
